(19) United States
(12) Patent Application Publication
(10) Pub. No.: US 2005/0220894 A1
(43) Pub. Date: Oct. 6, 2005

(54) Colostrum-based composition

(75) Inventors: Charles Edward Williams, Dunedin (NZ); Peter Graeme Hobman, Melbourne (AU); Simon Stephen Yarrow, Hamilton (NZ)

Correspondence Address: MCDERMOTT WILL & EMERY LLP, 600 13TH STREET, N.W., WASHINGTON, DC, 20005-3096, US

(73) Assignee: FONTERRA CO-OPERATIVE GROUP LIMITED, Auckland (NZ)

(21) Appl. No.: 11/136,575

(22) Filed: May 25, 2005

Related U.S. Application Data
(62) Division of application No. 10/416,831, filed on Oct. 8, 2003.

(30) Foreign Application Priority Data
Nov. 15, 2000 (NZ) 508234

(51) Int. Cl. A61K 35/20

(52) U.S. Cl. 424/535

(57) ABSTRACT

A composition including colostrum or a colostrum-derived product and hyperimmune milk (HIM) or a hyperimmune milk-derived product, in amounts sufficient to provide a combined spectrum of pathogen-binding activity against a broad-spectrum of pathogenic organisms.

### FIELD OF THE INVENTION

This invention relates to a colostrum-based composition having clinical application in the management of infection-associated disease, including gastrointestinal and joint diseases.

### BACKGROUND

Infection of the gastrointestinal tract is common, for example, with bacteria, viruses, yeast or parasitic pathogens which can evoke acute infection (e.g. gastroenteritis involving, for example, Salmonella, Shigella or acute viral infections) or chronic infections such as those involving Campylobacter jejuni, Clostridium difficile and Yersinia enterocolitica. Generally, the host's own immune system can deal with the presence of such pathogens. However, in some circumstances such pathogens, for example Yersinia enterocolitica and Helicobacter pylori, can become established in the stomach and bowel flora and cause serious disease.

In healthy animals infection with pathogenic micro-organisms is controlled through the immune system. Immunoglobulins play an integral role in the immune system. The most prevalent immunoglobulin in all species of animals is immunoglobulin G (IgG). Clinical trials have demonstrated that specific antibodies exist in bovine milk which are effective against both enteropathogenic and enterogenic organisms.

In all species of mammals immunoglobulins are passed from the mother to its young to provide passive immunity to a newborn animal. In humans and apes IgG and its compliment of antibodies pass across the placental barrier to the foetus during the second two-thirds of gestation. This passage appears to be selective in that other immunoglobulins are not transferred. In cattle, passive immunity is provided through the secretion of immunoglobulins, and in particular IgG, in colostrum produced by the cow in the first few days after birth of the calf, and the absorption of those immunoglobulins through the gastrointestinal tract of the calf. This absorption appears to be selective, in that there is a preferential passage of IgG through the gut wall, in comparison with other immunoglobulins.

Bovine milk antibodies have been shown to be an effective means of providing local protection within the gastrointestinal tract against disease caused by pathogenic micro-organisms. Trials have shown that specific antibodies in bovine milk are effective against enteropathogenic and enterotoxigenic Escherichia coli, cryptosporidium, rotavirus and Shigella flexneri.

Hyper Immune Milk (HIM) can be obtained from dairy cows that have been hyperimmunised by proprietary antigens to increase the concentration of specific antibodies (i.e titres) that are active against the chosen antigen. Such methods create unique milk products containing enhanced quantities of biologically functional antibodies and immune modulators. HIM is known to be beneficial for the prevention of certain diseases by fortifying the body's natural resistance to disease-causing antigens.

HIM is also recognised to contain components that appear to have anti-inflammatory activity and may have efficacy in the treatment of joint disease.

Gangliosides, particularly GM₃ and GD₃, are a key component of the plasma membrane of all human cells, and are particularly abundant in the nervous system. They play an important role in cell to cell recognition, cell signalling and cell growth. Gangliosides are polar complex phospholipids and comprise a ceramide backbone coupled to a sugar chain. The sugar chain contains an acidic sugar, N-acetyl-neuraminate (sialic acid).

Gangliosides are important components of human and bovine milk. At different times during lactation the proportion of different gangliosides in human breast milk varies. For example, GD₃ predominates initially, whilst GM₃ increases during lactation to become the main ganglioside after about one month. Gangioslides appear to have three major physiological functions; they block the effect of certain pathogens; they promote nerve cell growth and repair; and they may have a role in the regulation of cell growth and differentiation. Their ability to prevent the adherence of pathogens such as E. coli, rotavirus and Helicobacter pylori makes them of potential benefit in the prevention of enteric infections in the intestinal tract and as anti-ulcer agents. Idota et al, in Biosci, Biotech, Biochem. 59(1): 69-72 (1995) demonstrated that gangliosides GM₃ and GD₃ reduced the binding of E. coli to human intestinal cells.

The high concentration of gangliosides in the human brain and their effects on nerve cell growth make them of potential benefit in promoting learning and for recovery from stroke and Alzheimer's disease. Lower concentrations of gangliosides in the brain have been associated with irreversibly impaired learning behaviour in rats, for example. Further, the exogenous administration of gangliosides has been shown to provide partial protection against experimental allergic neuritis, and gangliosides accelerate nervous system repair in vivo in a cat model. In terms of cell growth/development, ganglioside GM₃ binds to epidermal growth factor (EGF) and inhibits EGF-dependent receptor tyrosine autophosphorylation and cell growth. Decreased levels of ganglioside expression were associated with increased EGF receptor autophosphorylation on tyrosine residues and increased EGF-stimulated cellular proliferation.

There is also evidence to suggest that O-acetylated GM₃ may enhance immunological activity of intact tumour cells. Gangliosides are potent pharmacological regulators of cell growth and differentiation. The direct addition of gangliosides to tissue culture medium causes growth inhibition by extending the length of the G1 phase of the cell cycle and blocks cellular proliferation in the presence of fibroblast growth factor and platelet-derived growth factor.

Bovine colostrum is also known to be rich in other nutritionally important and biologically active components, such as growth factors (that have been shown in numerous scientific studies to assist with skin and microscopic tissue/muscle healing and repair), antimicrobial substances (e.g. lactoferrin), minerals and vitamins. Some of the major growth factors and other proteins in bovine colostrum are shown in Table 1 below.

TABLE 1
Growth Factors

| | | |
|---|---|---|
| IGF-1 | 50-2000 | ng/ml |
| IGF-2 | 200-600 | ng/ml |
| TGF-beta 1 | 18 | ng/g |
| TGF-beta 2 | 863 | ng/ml |

Immune/Antibacterial enzymes

| | | |
|---|---|---|
| IgG1 | 52-87 | g/L |
| IgG2 | 1.6-2.1 | g/L |
| IgM | 3.7-6.1 | g/L |
| IgA | 3.2-6.2 | g/L |
| Lactoferrin | 1.5-5 | mg/MI |
| Lysozyme | 0.14-0.7 | mg/ |
| Lactoperoxidase | 11-45 | mg/L |

Hyper Immune Colostrum can be obtained from cows in a similar manner to that used to produce HIM, as discussed previously.

Arthritis is a degenerative condition involving degeneration of the joints and connective tissue, marked by pain and swelling. Synovial membranes (connective tissue) thicken and the joints swell and are red and tender.

There are numerous regulatory and growth factors present in colostrum which offer anti-inflammatory protection. This protection may be in part due to the elimination and/or neutralisation of microbial pathogens which are known to be associated with secondary diseases such as some forms of arthritis.

The traditional approach to the management of disorders of the gastrointestinal tract, inflammatory disease and bone disease relies on the use of pharmaceutical compositions, with their associated potential side effects.

It is an object of the present invention to provide a composition which reduces or overcomes at least some of the abovementioned problems or which will at least provide the public with a useful alternative. One particular object may be to produce a nutritional-based composition including a combination of components selected to have a functional profile of benefit in the management of infection-associated disease including gastrointestinal, inflammatory or bone disease.

Other objects of the present invention may become apparent from the following description which is given by way of example only.

### SUMMARY OF THE INVENTION

According to one aspect of the present invention there is provided a composition including colostrum or a colostrum-derived product and hyperimmune milk or a hyperimmune milk-derived product, in amounts sufficient to provide a combined spectrum of pathogen-binding activity against a broad-spectrum of pathogenic organisms.

Preferably, the composition further includes a ganglioside, in an amount sufficient to provide anti-microbial binding activity.

Preferably, the composition further includes other milk lipids and/or milk carbohydrates or milk carbohydrate derivations.

In a further preferred form, the composition further includes calcium in an amount sufficient to provide the recommended daily requirement for bone health.

Preferably, the calcium is milk derived calcium.

Preferably, the composition includes by weight between about 50% and 95% colostrum, 5% and 50% HIM and 0% and 10% milk lipids leg gangliosides) and other components, wherein the amount of HIM plus milk lipid/other compounds cannot exceed 50% of the total composition.

In one preferred form the composition includes substantially 50-93% colostrum or colostrum-derived product, 545% hyperimmune milk or hyperimmune milk-derived product, 2-4% ganglioside-containing component and 0.1-1% calcium.

Preferably, the colostrum or colostrum derived product is present in an amount of 60% and the HIM or HIM derived product at 35%, ganglioside component 3% and calcium 1.5%

Preferably, the ganglioside-containing component includes sufficient gangliosides to provide a dosage of 5-50 mg/day.

Preferably, the colostrum or colostrum-derived product is bovine colostrum powder.

Preferably, the colostrum powder is phospholipid coated.

Preferably, the colostrum is hyper immune colostrum.

Preferably, the hyperimmune milk or hyperimmune milk-derived product is bovine hyperimmune milk protein powder or skim milk powder.

Preferably, the ganglioside-containing component is derived from bovine milk.

Preferably, the gangliosides include ganglioside GM₃ and GD₃.

In another preferred form the composition includes substantially 65-70% colostrum milk protein powder, substantially 24-30% hyperimmune milk powder, substantially 24% ganglioside-containing component and substantially 0.5-1.5% milk calcium.

According to a further aspect of the present invention there is provided a composition derived from milk and/or colostrum, including colostrum or colostrum-derived product, hyperimmune milk or hyperimmune milk-derived product and gangliosides, in proportions selected to provide a functionally balanced composition and enhanced anti-inflammatory activity.

Preferably the composition includes substantially 50-93% colostrum or colostrum-derived product, substantially 5-45% hyperimmune milk or hyperimmune milk-derived product and substantially 2-4% ganglioside-containing component.

Preferably, the composition further includes milk calcium. Preferably, in a proportion of substantially 1.5%.

Preferably, the ganglioside-containing component includes sufficient gangliosides to provide a dosage of 5-50 mg/day.

According to a further aspect of the present invention there is provided a method of treatment of an infection-associated disease or prophylaxis against an infection-associated disease, using a composition including colostrum or a colostrum-derived product and hyperimmune milk or a hyperimmune milk-derived product in amounts sufficient to provide a combined spectrum of pathogen-binding activity against a broad-spectrum of pathogenic organisms.

Preferably, the composition further includes gangliosides and calcium.

Preferably, the composition includes substantially 55-95% colostrum or colostrum-derived product, 5-45% hyperimmune milk or hyperimmune milk-derived product, 2-4% ganglioside-containing component and 0.1-1% calcium.

Preferably, the infection-associated disease is an H. pylori or Clostridium difficile associated disease.

Alternatively, the infection-associated disease is irritable bowel syndrome or disease, or an arthritic condition.

According to a further aspect of the present invention there is provided the use of a composition including colostrum or a colostrum-derived product and hyperimmune milk or a hyperimmune milk-derived product, in the manufacture of a composition for the management of an infection-associated disease.

Preferably, the colostrum or colostrum-derived product and hyperimmune milk or hyperimmune milk-derived product is included in amounts sufficient to provide a combined spectrum of pathogen-binding activity against a broad-spectrum of organisms.

Preferably, the composition further includes gangliosides and calcium.

Preferably, the infection-associated disease is an H. pylori associated disease, irritable bowel syndrome or an arthritic condition.

According to a further aspect of the present invention there is provided the use of a composition including colostrum or a colostrum-derived product and hyperimmune milk or a hyperimmune milk-derived product in the manufacture of a composition for use in the management of an inflammatory disease.

Preferably the colostrum or a colostrum-derived product and hyperimmune milk or a hyperimmune milk-derived product is included in proportion selected to provide a functionally balanced composition and enhanced anti-inflammatory activity.

Preferably, the composition further includes gangliosides and calcium.

Preferably, inflammatory disease is an arthritic condition.

According to a further aspect of the present invention there is provided a composition substantially as herein described and with reference to the accompanying examples.

Other aspects of the present invention may become apparent from the following description which is given by way of example only.

### BRIEF DESCRIPTION OF THE FIGURES

FIG. 1: Shows a comparison of the in vitro binding of various compositions to Candida albicans;

FIG. 2: shows a comparison of the in vitro binding of various compositions to Salmonella typhimurium;

FIG. 3: shows a comparison of the in vitro binding of various compositions to Klebsiella pneumoniae;

FIG. 4: shows a comparison of the in vitro binding of various compositions to Clostridium difficile;

FIG. 5: shows a comparison of the in vitro binding of various compositions to Escherichia coli 0157;

FIG. 6: shows a comparison of the in vitro binding of various compositions to Helicobacter pylon.

FIG. 7: shows a further comparison of the in vitro binding of various compositions to Candida albicans.

FIG. 8: shows a further comparison of the in vitro binding of various compositions to Salmonella typhimurium.

FIGS. 9-11: show compositions against a theoretical calculation.

### DETAILED DESCRIPTION OF INVENTION

The invention is generally directed to the provision of a composition that has application in the management of a variety of infection-associated diseases, including gastrointestinal and joint diseases. The management may be prophylactic or may be in response to existing symptoms.

It has been found that by combining colostrum and HIM (or products derived from these) an effect on a broad range of infection causing bacteria is observed that exceeds the effect the individual components have when used alone. In some bacterial cases, the effect is greater than either individual component and in others, the effect is the same or similar when using significantly lower amounts of the colostrum/HIM components in the composition.

A particularly preferred additive to the composition is a ganglioside containing component. Gangliosides are milk lipids and the health benefits of such a component, particularly if GM₃ or GD₃ enhanced, have been discussed previously herein.

Other additions include calcium, particularly milk derived, and other milk lipids, phospholipids and milk carbohydrates and derivatives.

In a preferred form the composition should not include more than 50% by weight of HIM (plus other components if any) with the balance being made up of colostrum or colostrum-derived product. It is preferred that the amount of colostrum in the product is between 50% and 95% by weight, more preferably between 60% and 80% and most preferably between 60% and 75% by weight. The HIM should be present between 5% and 50%, more preferably between 10% and 45%, and most preferably between 15% and 40%. Of course, other combinations of ranges could also be used.

Colostrum was derived from healthy non-immunised pasture-fed cows, separated from the cream, pasteurised and spray dried to form a protein powder. The use of colostrum from hyperimmunised cows (ie hyper immune colostrum) is also an option. In a further preferred form the colostrum powder was coated with milk phospholipids, the beta-lipid coating including, in particular: phosphatidylcholine, phosphatidylserine, phosphatidylethanolamine and sphingomyelin.

Bovine colostrum has been demonstrated to show specific binding against many pathogens, including Candida albicans, E. Coli 0157, Helicobacter pylori, Propionibacterium acnes, Klebsiella pneumoniae, Staphylococcus aureus, Streptococcus pyogenes, Streptococcus mutans, Yersinia entercolitica, Staphylococcus epidermidis, Salmonella typhimurium, Salmonella enteritidis, Haemophilus influenzae, Campylobacter jejuni, and Listeria monocytogenes.

In addition, bovine colostrum is rich in vitamins, minerals, growth factors (eg IgG) and immune/antibacterial enzymes. Use of hyperimmune colostrum will maximise the content of biologically functional antibodies and immune modulators present in the colostrum.

Colostrum also contains numerous different components which offer anti-inflammatory properties, including lactoferrin, proline-rich polypeptide, lysozyme and growth factors (eg IgG), some of which also have anti-microbial activity. In addition, the bovine colostrum employed in the composition of the present invention has an immunomodulatory effect by blocking pro-inflammatory cytokines such as TNF-alpha and IL-6.

Immune milk was obtained from dairy cows hyperimmunised by proprietary antigens and methods that create unique milk products that contain enhanced quantities of biologically functional antibodies and immune modulators.

The immunoglobulins in HIM have been shown to specifically bind the following pathogens: Pseudomonas aenrginosa, Aerobacter aerogenes, Haemophilus typhimurium, Streptococcus mitis, Proteus vulgaris, Shigella dysenteriae, Diplococcus pneumoniae, Actinomyces (fungus), Streptococcus sanguis, Streptococcus salvarius, Streptococcus pyogenes (types 1,3,5,8,12,14,18,22).

These pathogens are known to be contributory factors in many disease states or physiological disorders, including food poisoning, gastrointestinal ulcers, skin and lung infection, thrush, dental caries and gum disease, surgical infections, respiratory tract disease, bowel disease and haemorrhagic disease.

The following list correlates typical pathogen and the diseases related to them. The composition according to the present invention can be used to treat and/or control such occurrences.

TABLE 1

| Pathogen | Causes Disease |
|---|---|
| Candida Albicans | Candidiasis, thrush |
| E. coli 0147H7 | Food poisoning |
| Clostridium difficile | Digestive disease |
| Enterobacter aerogenes | Skin and lung infection. Species |
| | also found in large intestine |
| Yersinia enterocolitica | Digestive disease |
| Proteus vulgaris | Possible bowel disease |
| Sheigella Flexneri | Possible digestive disease |
| Salmonella typhimurium | Food poisoning |
| Bacterioides thetaiomicronin | Species found in large intestine |
| Bacteriodes fragilis | Species found in large intestine |

Gangliosides, particular GM₃ and GD₃, as discussed above, have been shown to have a range of health benefits, relating to gut health, brain health and cell growth/development. It is possible that their anti-adherence/anti-microbial (e.g. with E. coli and H. pylori and various microbial toxins such as Shigra toxin) activity may further enhance or supplement the clinical benefits of colostrum combined with HIM.

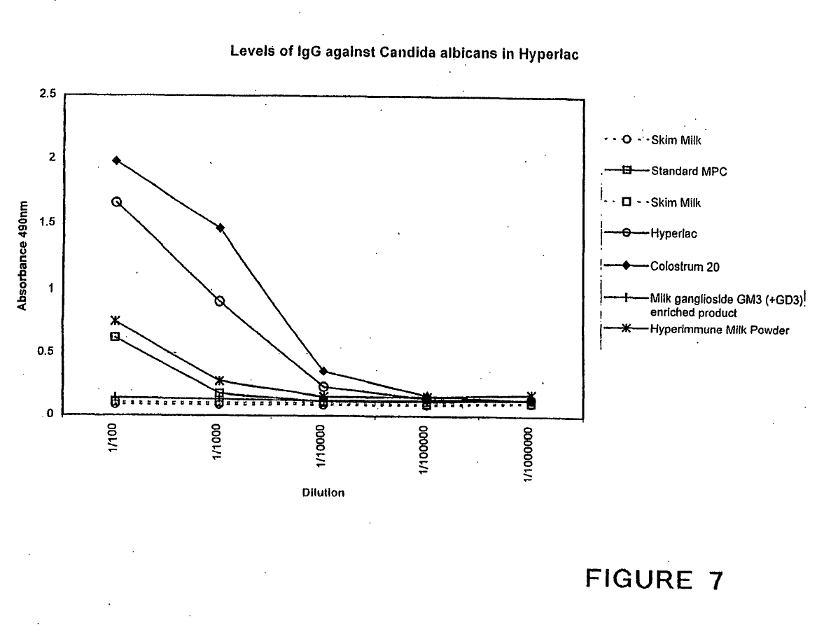

The ELISA graphs (see FIG. 7 herein) show that the particular ganglioside product added to the present composition has no apparent binding effect on the selected micro-organisms on its own. Gangliosides have an antimicrobial effect but do not bind to the micro-organism. Rather the activity is understood to be an anti-adherence effect (see Idota et al above). Thus the action of the ganglioside component should not affect the micro-organism binding action of the HIM or colostrum. This suggests that the composition's effects occur without input from the ganglioside product, showing that the synergies in binding effect are created between the colostrum and HIM.

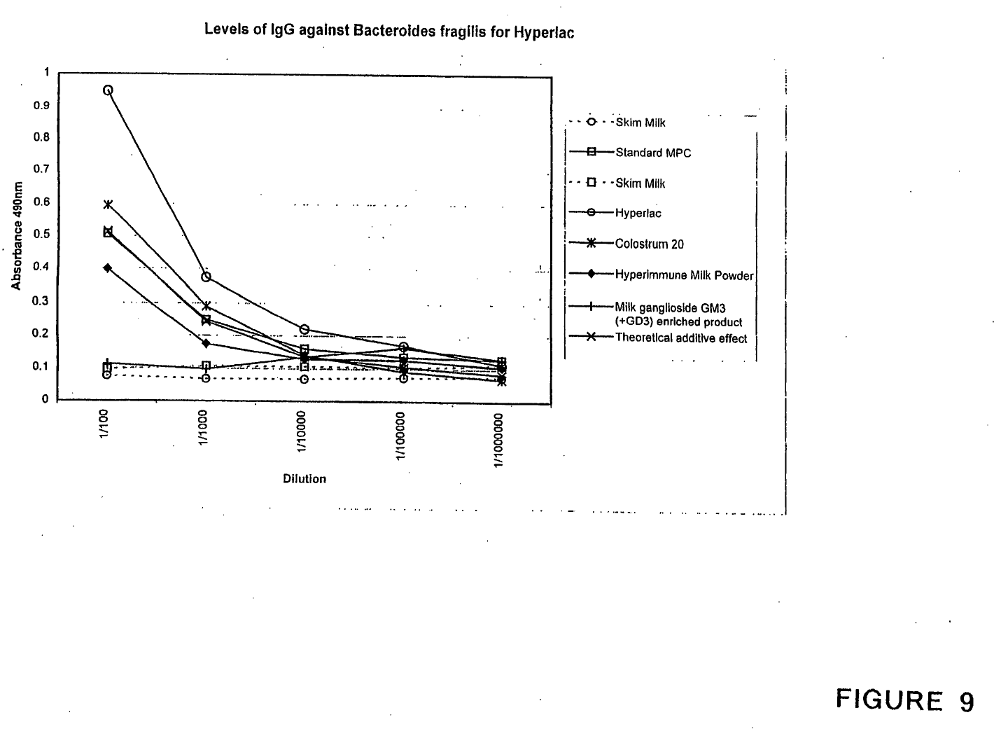

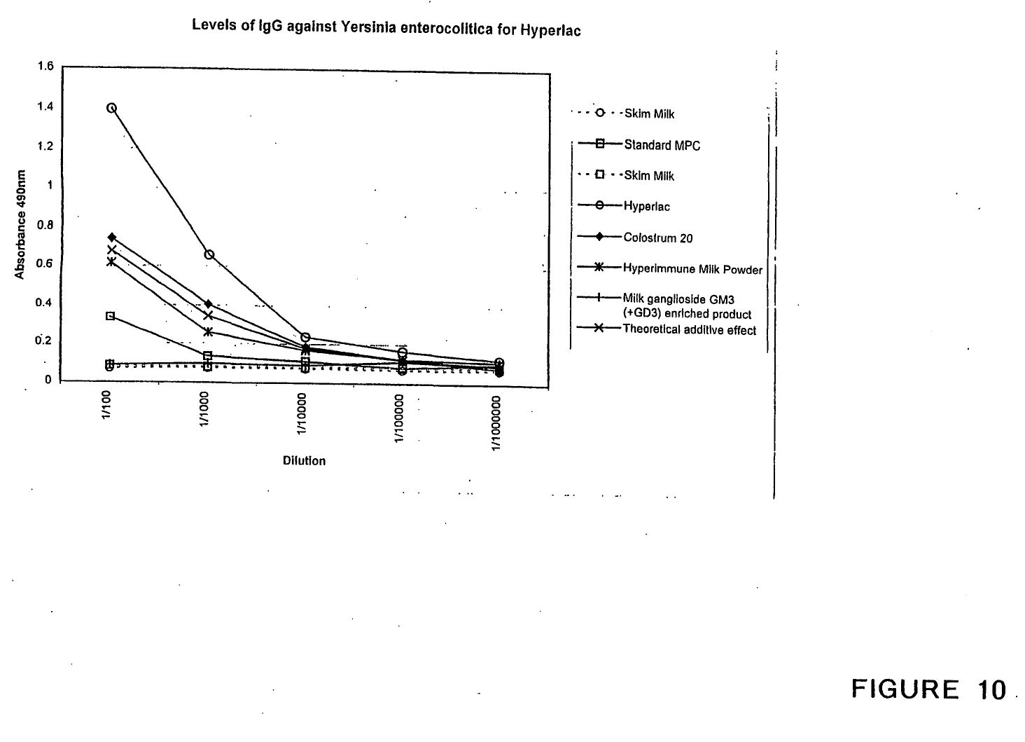

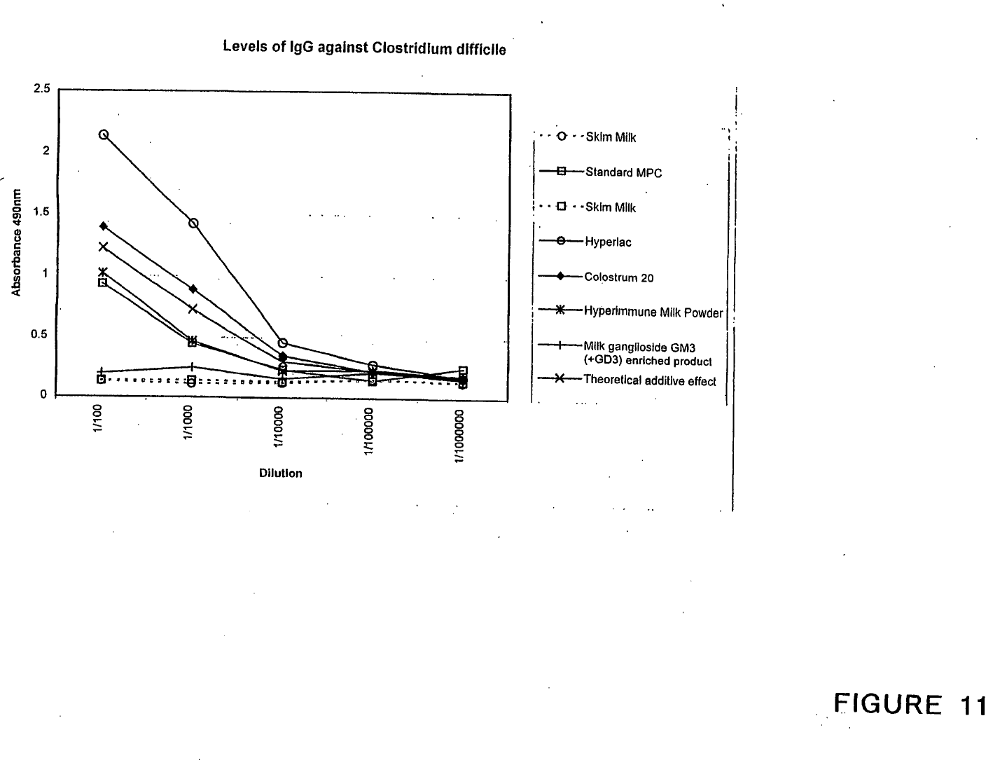

The following graphs (FIGS. 9-11) illustrate the binding effect that would be expected if the effect of combining the ingredients shown was purely additive. However, it can be seen in FIGS. 9-11 that in fact a greater binding effect has been achieved than would be expected, again supporting the synergistic aspect of the HIM and colostrum combination.

It is possible that the observed enhanced binding effect of the HIM and colostrum composition (as seen in the Figures) could be due to the effect of the ganglioside. Such an effect would be very unexpected and would, in itself, constitute a synergistic effect. To this extent the combination of HIM and colostrum (or derived products) together with a ganglioside constituting another aspect of the present invention.

To provide appropriate anti-microbial binding activity (and possible synergies) a concentration of gangliosides in a composition of the invention to achieve a daily dosage of 5 to 50 mg is preferred. The gangliosides are preferably present in an amount of between about 0.02% to 0.5% of the composition. The ganglioside containing component may therefore amount up to about 10% of the composition, depending on the amount of ganglioside in the component. More preferably the range will be between 2% to 4%.

A ganglioside product was prepared as a non-genetically modified product extracted directly from milk. The product (or ganglioside-containing component) contains concentrated gangliosides GM₃ and GD₃.

The composition can also contain other lipids and carbohydrates. Examples of lipids are: sphingomyelin, phophotidylserine, phosphotidylcholine. Examples of carbohydrates are: galacto oligosaccharides, sialyl lactose, sialated oligosaccharides. These examples are not intended to be limiting.

Calcium employed in the compositions of the invention was also milk-derived. The preferred content is between about 0.1% to 2% of the composition. More preferably between 0.1% to 1.5%.

The specific binding activity and complimentary action against gastrointestinal pathogens by colostrum and hyperimmune milk protein concentrates, further assisted by the sialic acid binding actions of gangliosides, means that compositions of the invention are targeted towards the treatment of acute and chronic gastrointestinal disease in particular. For example, gastric ulcers, duodenal ulcers, ulcerative colitis, Crohn's disease, chronic diverticulosis, irritable bowel disease, pseudomembranous colitis, antibiotic associated diarrhoea, travellers diarrhoea, juvenile diarrhoea, and cryptosporidiosis associated diarrhoea.

By combining colostrum and hyper-immune milk, in appropriate proportions, a product is produced which has a broad-spectrum of activity against pathogenic organisms, with both predictable and potentially unexpected clinical benefits. The bacterial action of the individual components is expected but the level of the activity of the combination is unexpected and offers therapeutic and cost advantages.

Typical composition ranges for the major components of the composition will be:

| Component | Minimum % | Maximum % |
|---|---|---|
| Colostrum | 50 | 95 |
| Hyperimmune Milk | 5 | 50 |
| Ganglioside/Other Components | 0 | 10 |

The total amount of HIM plus other components should not exceed 50% of the composition. As will be readily apparent the ranges are indicative only.

As will be shown in the examples and trials completed, the effects of treatment using a composition according to the present invention are significant. While the main individual components of the composition (HIM and colostrum) have known beneficial effects, it is unexpected that the combination would have the efficacy shown herein. To this extent it is hypothesised that there may be a synergistic interaction occurring between the HIM and colostrum components of the composition either directly or indirectly.

### EXAMPLE 1

A test composition was prepared including 70% colostrum milk protein powder, 24% hyperimmune milk powder, 4% ganglioside-containing component, whey powder, lactose and 1.5% milk calcium. Further details of the chemical makeup of the resultant composition are shown in Table 2.

TABLE 2
Composition of Trial Composition

| COMPOSITION | | m/m |
|---|---|---|
| Protein (d.b.) | | 76.0% |
| Fat | | 2.5% |
| Lactose | | 12.0% |
| Ash | | 7.5% |
| Moisture | | 4.9% |
| Immunoglobulin G (determined by | | >15% |
| HPLC-Protein G | | |
| Gangliosides | | 0.036% |
| Calcium | | 2.15% |
| IGF-1 | Min | 500 ± 50 ng/g |
| Sphingomyelin | Min | 0.0325% |
| Phosphatidyl choline | Min | 0.0600% |
| Phosphatidyl ethanolamine | Min | 0.0350% |
| Phosphatidyl serine | Min | 0.0075% |

In Vitro Binding Studies

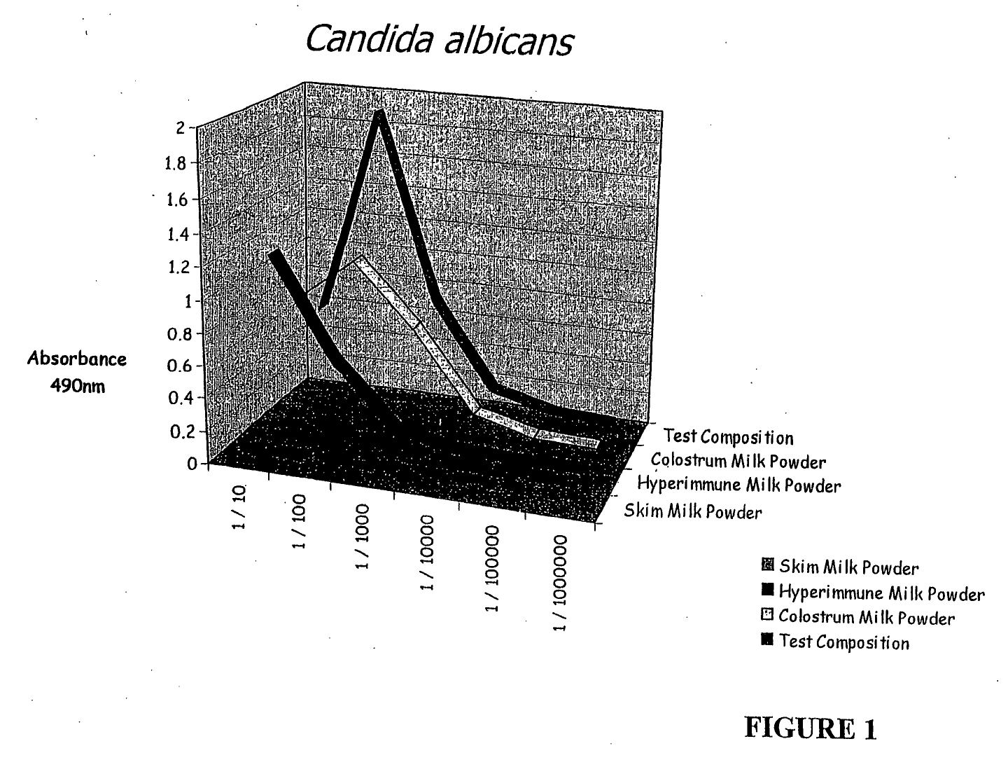

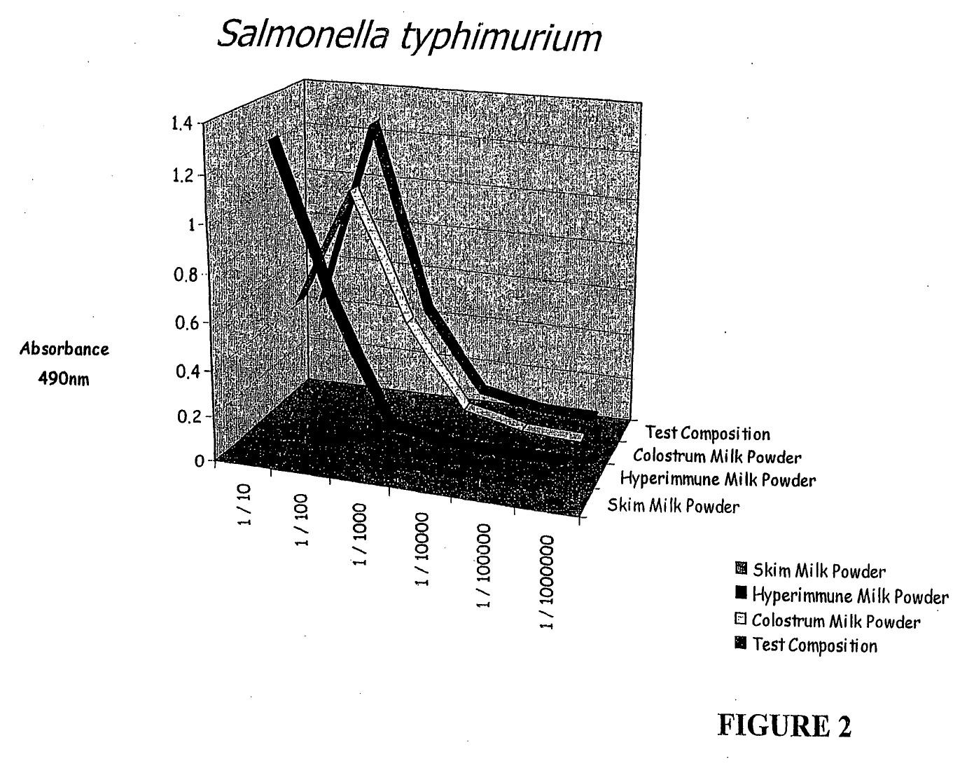

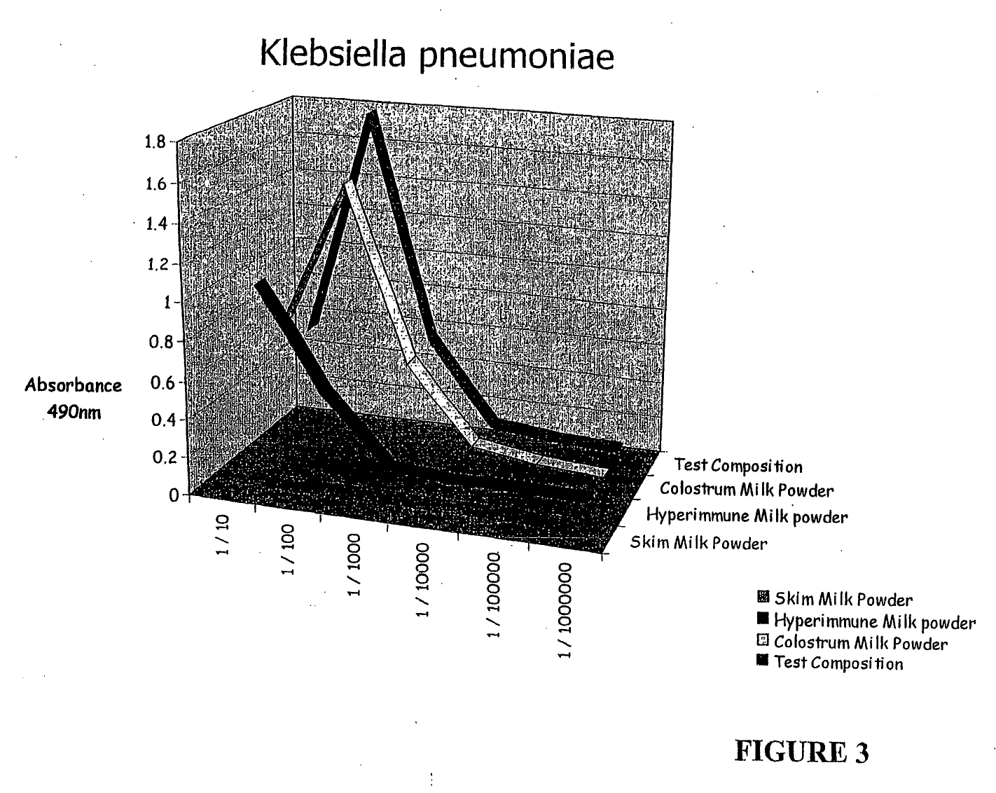

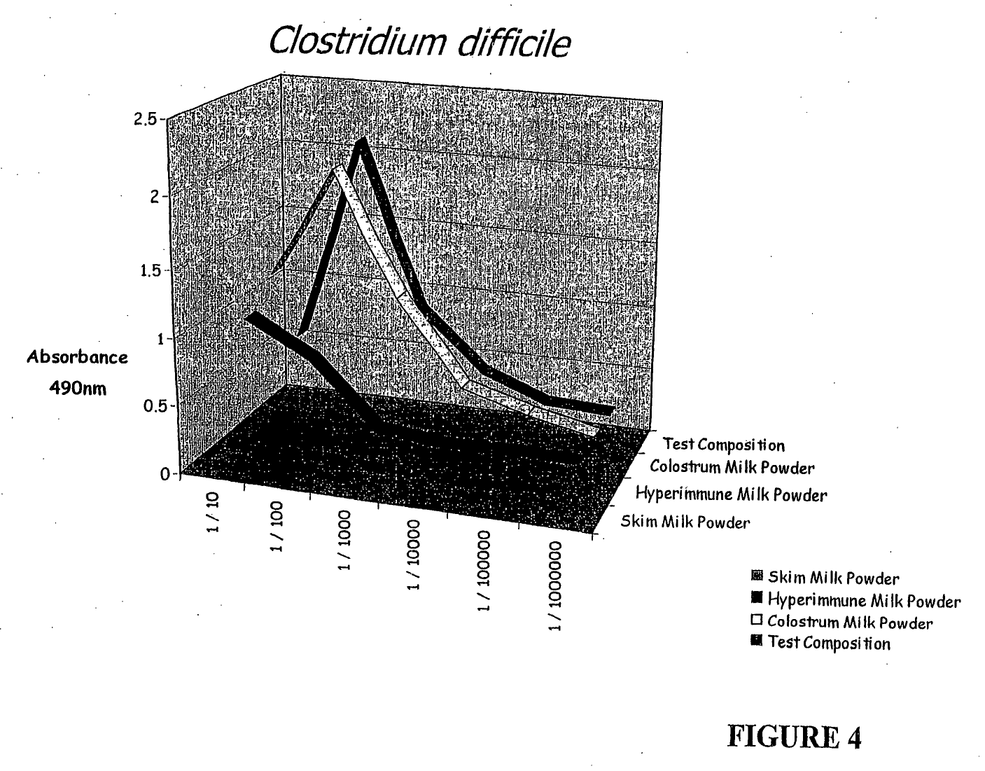

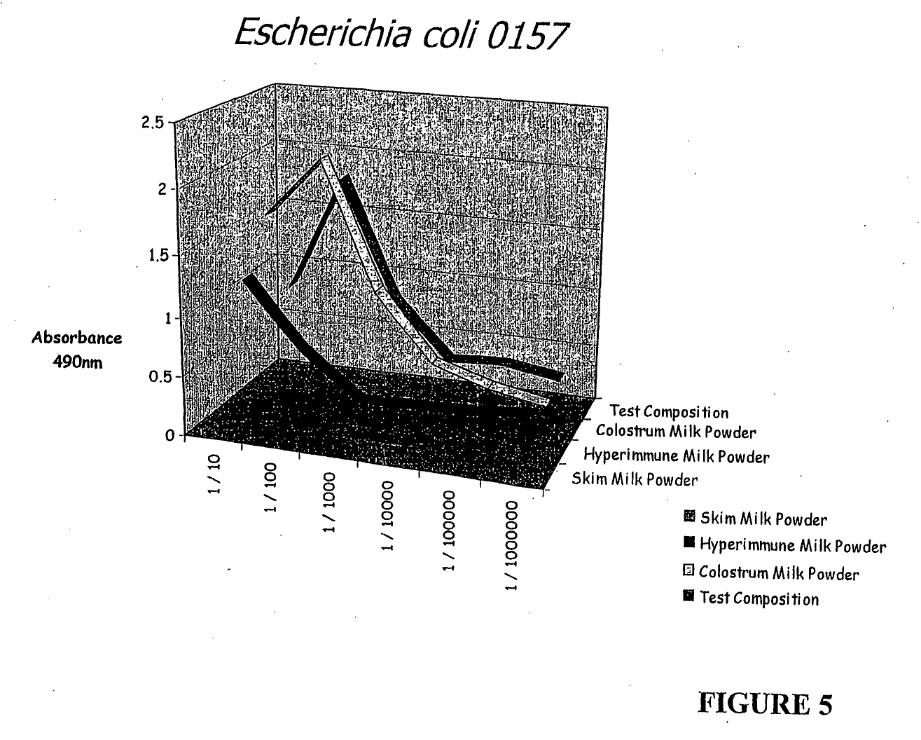

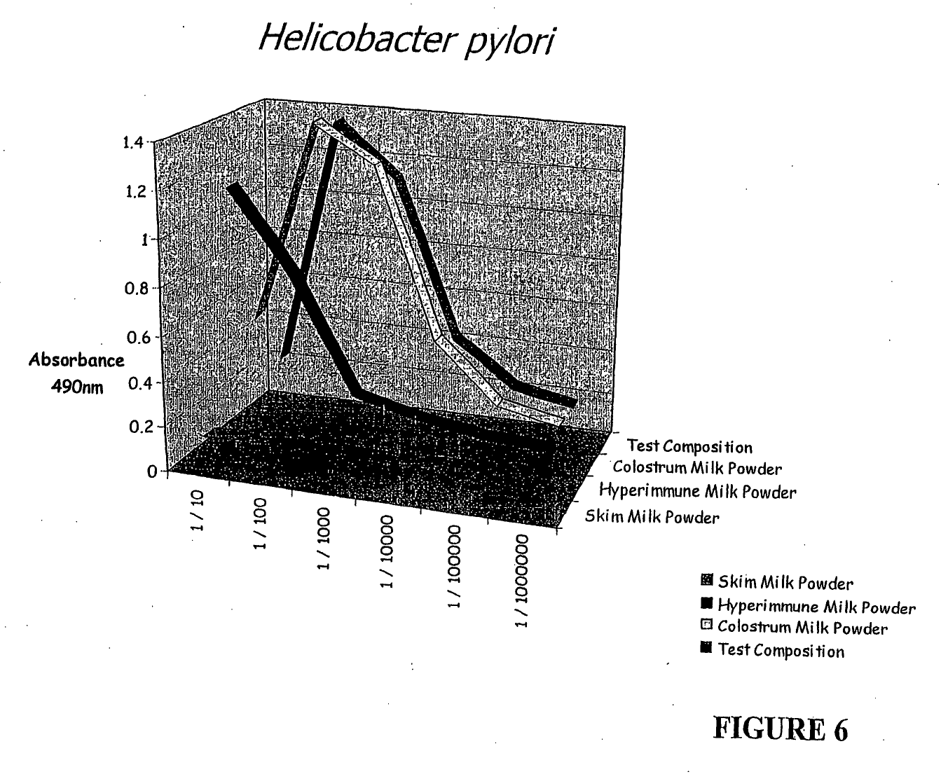

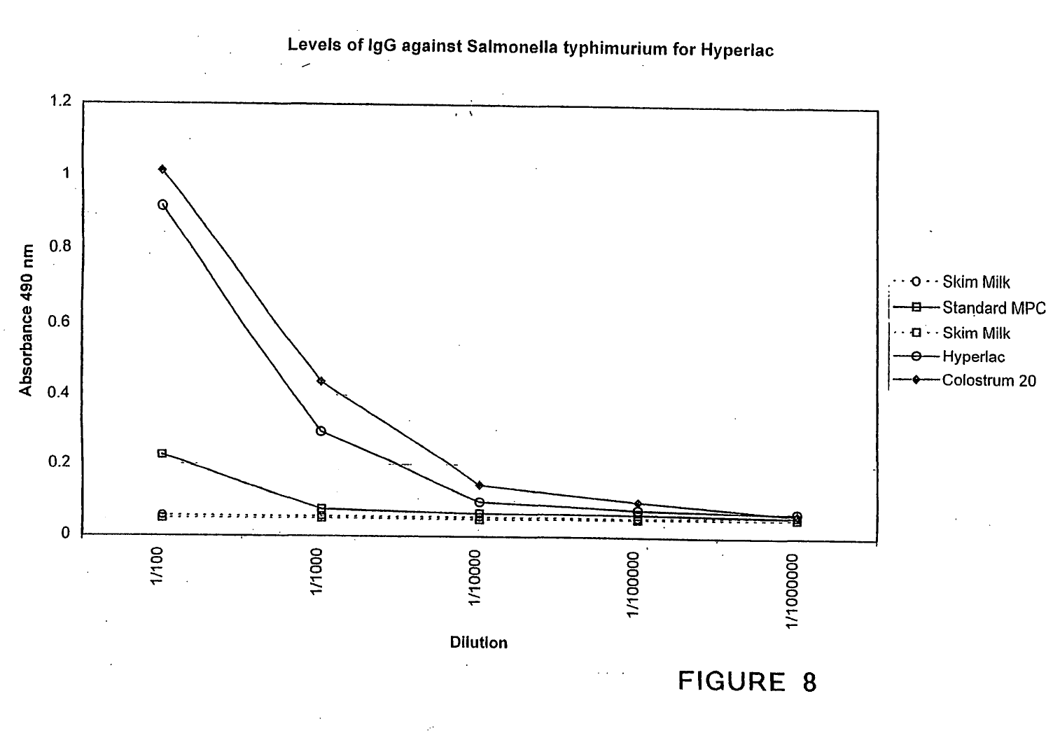

Binding studies were performed to compare the binding of the test composition, colostrum milk powder, skim milk powder and HIM with various bacterial and yeast pathogens including Candida albicans, Salmonella typhimunium, Helicobacter pylon, E. Coli spp Clostridium difficile and Klebsiella pneumonia. The results are shown in FIGS. 1-8. In summary, the test composition demonstrated a very high rate of specific binding with these species.

In Vitro Anti-Inflammatory Properties

The Tight Junction assay measures the degree of leakiness of a monolayer of epithelial cells, particularly kidney cells. A normal characteristic of epithelium, like the intestine or kidney, is that the cells form a tight barrier, but this barrier function is sometimes compromised making the epithelium leaky. Such is the situation occurring during inflammation.

In the assay, cells are incubated with or without a test sample and are then challenged with a compound to make them leaky. Any increase in leakiness is measured and activity reported as change from the baseline prior to challenge. The lower the activity unit the less leaky the cells become and hence the more protective the sample.

The activity of HIM in this assay has been compared with its anti-inflammatory properties in various mouse models of inflammation, and a good correlation found. Thus, the Tight Junction assay provides a good screening assay for anti-inflammatory activity in milk samples.

The test composition was compared in this assay with HIM, bovine colostrum and a control. Each of the samples were made to 10% (w/v) with phosphate buffered saline and then centrifuged at 100,000 g for one hour to remove caseins. The whey fraction was tested in the Tight Junction assay. MDCK cells were grown to confluence on Transwell and the whey was added at 10% for 48 hours. Control cells had no whey added. Transepithelial electrical resistance across the cell monolayer was measured before and one hour after challenge with EGTA. All samples were tested in quadruplicate.

The results are shown in Table 3.

TABLE 3

| Sample | Activity (units) |
|---|---|
| Bovine colostrum | −5 |
| Test composition | −11 |
| Hyperimmune milk | −13 |
| Control | 35 |

In comparison with the control, each of the samples had significant protective activity. The activity of HIM was significantly greater than that of bovine colostrum. The test composition was statistically significantly superior to the bovine colostrum, and only a little less effective than the HIM, notwithstanding that the test composition included only 24% hyperimmune milk. It seems clear that the test composition has increased the protective activity of the hyperimmune milk on a per unit basis. This increase is considered significant.

In summary, the test composition of the invention includes a combination of ingredients each of which has particular anti-microbial binding and/or anti-inflammatory activity which may combine to produce particular and unexpected clinical benefits in a broad range of diseases, including infection-associated diseases, and particularly gastrointestinal, inflammatory and bone related disorders. Such benefits are an unexpected result of the combination used.

Furthermore, the composition may include sufficient calcium for the average required daily human intake; and includes a broad range of vitamins and minerals, and has a balanced protein/carbohydrate mix. The composition may be of benefit for the prophylaxis or treatment of disease, alone or in combination with conventional pharmaceuticals.

Another typical chemical makeup of the composition according to the invention is as shown in Table 4.

TABLE 4

| Composition | | Typical | % m/m |
|---|---|---|---|
| Protein (d.b.) | | 77 | % |
| Fat | | 2 | % |
| Lactose | | 13 | % |
| Ash | | 7 | % |
| Moisture | | 5 | % |
| Immunoglobulin G (determined | | 17 | |
| by HPLC-Protein G) | | | |
| IGF-1 | min | 500 ± 50 | ng/g |
| Calcium | | 2.2 | % |
| Gangliosides | | 0.079 | % |
| Sphingomyelin | | 0.28 | % |
| Phosphatidyl choline | | 0.0600 | % |
| Phosphatidyl ethanolamine | min | 0.0350 | % |
| Phosphotidyl serine | min | 0.0075 | % |

The compositions of Tables 2 and 4 show a composition produced using milk protein concentrate versions of the composition. As will be readily apparent to the skilled person, skim milk versions can also be used.

### EXAMPLE 2

With reference to FIGS. 1-8, a comparison of the test composition with a control (skim milk powder) and the two major components of the test composition, are shown. The Figures show the effects of the various samples in in vitro binding of a variety of pathogens.

As can be seen, the test composition shows significant benefits in comparison to the other samples. This is surprising because the test composition contains significantly less colostrum and HIM than is present in the respective comparative samples.

With reference to FIG. 7 it can be seen that a ganglioside sample has little or no binding effect on the pathogen Candida albicans. This has been discussed previously herein.

### EXAMPLE 3

A pilot study was conducted by a gastroenterology clinic concentrating on Coeliac disease and IDB (Chrohns disease and ulcerative colitis). It involved 20 patients who were not responding to current therapies. The time period of study was 6-8 weeks. Dosage was 20 g twice daily. More than 45% were dramatically improved. More than 34% were clinically assessed as achieving therapeutic cure after recurrence/relapse with previous standard therapy.

Assessment of symptoms before and after consuming a composition according to the invention containing 60% colostrum, 35% HIM. 3% ganglioside containing component, and 1.5% milk calcium:

| Before | After |
|---|---|
| Oesophageal and lower bowel | Regular and normal stools with |
| camping/spasms | decreased frequency |
| Skin lesions | Normal sleep pattern |
| Severe constipation or | Lack of pain |
| diarrhoea | Higher energy/mood |
| Fatigue | improvement |
| High frequency of bowel | Decreased reliance on other |
| movement | medication |
| Pain | |
| Insomnia or lack of sleep | |

The composition of the present invention may be formulated in a tablet or capsule, or may be supplied in powder form for administration as a beverage. Depending on whether the product is to be used for prophylaxis or as treatment, and depending on the nature of the specific indication, it is envisaged that the effective dosage range may be from about 1 g to 40 g per day. In the management of gastrointestinal disorders, a preferred dosage regimen may preferably be in the range 10-30 g per day, with administration on a twice daily basis, on an empty stomach. Where the composition is used for stomach ulcers it is anticipated that it should preferably be administered in combination with a mucolytic agent. Use of the composition may enable a reduction in dosage or elimination of a conventional anti-ulcer medication.

Where in the foregoing description reference has been made to specific components or integers of the invention having known equivalents then such equivalents are herein incorporated as if individually set forth.

Although this invention has been described by way of example and with reference to possible embodiments thereof it is to be understood that modifications or improvements may be made thereto without departing from the scope or spirit of the invention as defined in the appended claims.

1-17. (canceled)

18. A method of treatment of an infection-associated disease or of prophylaxis against an infection-associated disease, using a composition including colostrum or a colostrum-derived product and hyperimmune milk or a hyperimmune milk-derived product in amounts sufficient to provide a combined spectrum of pathogen-binding activity against a broad-spectrum of pathogenic organisms.

19. The method according to claim 18 wherein the composition further includes gangliosides and calcium.

20. The method according to claim 18 wherein the composition includes substantially 55-95% colostrum or colostrum-derived product, 5-45% hyperimmune milk or hyperimmune milk-derived product, 2-4% ganglioside-containing component and 0.1-1% calcium.

21. The method according to claim 18 wherein the infection-associated disease is a H. pylori associated disease or irritable bowel syndrome or disease, or an arthritic condition.

22. The use of a composition including colostrum or a colostrum-derived product and hyperimmune milk or a hyperimmune milk-derived product, in the manufacture of a medicament for the management of an infection-associated disease.

23. The use according to claim 22 wherein the colostrum or colostrum-derived product and hyperimmune milk or hyperimmune milk-derived product are included in amounts sufficient to provide a combined spectrum of pathogen-binding activity against a broad-spectrum of organisms.

24. The use according to claim 22 wherein the composition further includes gangliosides and calcium.

25. The use according to claim 22 wherein the infection-associated disease is an H. pylori associated disease, or irritable bowel syndrome or an arthritic condition or a Clostridium difficile associated disease.

26. The use according to claim 23 wherein the colostrum is hyperimmune colostrum.

27. The use of a composition including colostrum or a colostrum-derived product and hyperimmune milk or a hyperimmune milk-derived product in the manufacture of a medicament for use in the management of an inflammatory disease.

28. The use according to claim 27 wherein the colostrum or a colostrum-derived product and hyperimmune milk or a hyperimmune milk-derived product are included in proportion selected to provide a functionally balanced composition and enhanced anti-inflammatory activity.

29. The use according to claim 27 wherein the composition further includes gangliosides and calcium.

30. The use according to claim 27 wherein the inflammatory disease is an arthritic condition.

31. The use according to claim 27 wherein the colostrum is hyperimmune colostrum.

32-36. (canceled)
